(19) United States
(12) Patent Application Publication
(10) Pub. No.: US 2012/0058150 A1
(43) Pub. Date: Mar. 8, 2012

(54) Methods for Delivering Compositions by Electrospraying a Medical Device

(75) Inventors: David A. Prawel, Loveland, CO (US); Susan P. James, Bellvue, CO (US); Ketul C. Popat, Fort Collins, CO (US)

(73) Assignee: COLORADO STATE UNIVERSITY RESEARCH FOUNDATION, Fort Collins, CO (US)

(21) Appl. No.: 13/041,960

(22) Filed: Mar. 7, 2011

Related U.S. Application Data
(60) Provisional application No. 61/310,780, filed on Mar. 5, 2010.
(60) Provisional application No. 61/329,464, filed on Apr. 29, 2010.

(51) Int. Cl. A61K 9/00; A61K 31/685; A61P 31/04; A61M 5/142; A61P 7/02; B05D 1/02; B05D 7/00; A61K 31/7036; A61P 35/00

(52) U.S. Cl. 424/400; 514/42; 514/78; 514/114; 427/2.1; 604/151; 604/891.1

(57) ABSTRACT

Methods are provided for administering a phospholipid composition to a subject, comprising coating a medical device with at least one layer of a phospholipid composition, wherein the coating is achieved by electrospraying the device with the composition, and wherein the composition is carrying or can carry at least one therapeutic agent.

### RELATED APPLICATIONS AND INCORPORATIONS BY REFERENCE

This application claims priority to U.S. Provisional Patent Application Ser. No. 61/310,780, filed on Mar. 5, 2010, and to U.S. Provisional Patent Application Ser. No. 61/329,464, filed on Apr. 29, 2010, which are incorporated herein by reference in their entirety.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### TECHNICAL FIELD

This invention relates to a method(s) for coating a medical device with at least one layer of a phospholipid composition, wherein the coating is achieved by electrospraying the device with the composition, and wherein the phospholipids are carrying or can carry at least one therapeutic agent.

### BACKGROUND

There are many causes of implant failure. Infection is one of the most serious. Infection rates resulting from surgical implantation of orthopedic devices occurs in approximately 0.5%-6% of cases (Soundrapandian, D., et al. 2007 Critical Reviews in Their Drug Carrier Systems 24(6):493-545), despite adherence to strict antiseptic operative procedures and the use of prophylactic antibiotics.

Chronic osteomyelitis often results from pathogens introduced from the skin into bony tissues during traumatic events, orthopedic surgeries repairing fracture or joint replacements (1-13% of cases), by oncological orthopedic surgeries (0-33% of cases), or any orthopedic surgery for patients suffering from diabetes (up to 13% of surgical cases) (Grimer, R J., et al. 2002 Clin Ortho and Related Res 395:193-203).

Implant loosening from the implant site may also result in implant failure. Loosening is often a result of poor binding of the mineral phase of the bone to the metal implant surface and the presence of non-mineralized tissue at the bone-implant interface (Steflik, D. E., et al. 1998 J Biomed Materials Res 39(4):611-620; Merolli, A., et al. 2006 J Materials Sci: Materials in Med 17(9):789-794). Roughly a third of total joint replacements fail due to particulate wear debris, poor apposition and osseointegration, resulting in loosening of the device.

Infections alone, as they relate to orthopedic devices, result in billions of dollars in health care costs annually in the United States, and 3 times higher worldwide. The overall annual costs of infections relating to orthopedic implant in the United States range between 150 and 200 million USD (Sculo, T. 1993 AAOS Instr Course Lect 42, Am Acad Orthoped Surg; Wei, M., et al. 2010 J Arthroplasty 25(6):906-912 e901) and about 3 times worldwide.

Coatings and coating techniques that provide adherent, mechanically stable, biological fixation and close apposition between bone and an implant can improve osseointegration of an implant device, leading to longer service life for implanted devices, greater long-term surgical success rates and better quality of life for patients.

Dip and drip coating are common techniques for applying phospholipids to titanium, particularly in studies of cell-related phenomenon. Dip coating (Willumeit, R., et al. 2007 European Cells & Mater 13:11-24) is performed by dipping a titanium sample in a bath of one or more phospholipids dissolved in a solvent. However, dip coating techniques are difficult to control or quantify as to the amount of phospholipid material actually deposited on the titanium surface without destructive testing.

Drip coating (Bosetti, M., et al. 2005 Biomater 26:7573-7578; Santin, M., et al. 2005 J R Soc Interface 3:277-281) entails dripping solutions containing one or more phospholipids onto titanium test samples. The main disadvantage of this technique is that the drip process creates thick, discontinuous, non-adherent coatings which often form 3-dimensional gels in simulated body fluid, resulting in instability under mechanical stresses (Hui, S. W., et al. 1981 Science 212(4497):921-923).

Numerous other coating techniques have emerged, but these are limited by complex processing requirements, toxic chemicals used in the process, poor control of coating texture, composition, and adhesion, and long reaction times (Berkland, C., et al. 2004 Biomater 25(25):5649-5658).

Electrohydrodynamic atomization (electrospraying or E-spraying) is a versatile method of creating thin, adherent coatings by atomizing a liquid by means of electrical forces (Cloupeau, M., et al. 1989 J Electrostatics 22(2):135-159; Grace, J. M., et al. 1994 J Aerosol Sci 25(6):1005-1019). This technique has seen little application in biomedical sciences.

A thorough review on electrospraying and its different modes is provided by Cloupeau and Prunet-Foch (Cloupeau, M., et al. 1989 J Electrostatics 22(2):135-159).

The present invention is directed toward overcoming one or more of the problems discussed above.

### BRIEF SUMMARY OF THE INVENTION

In one aspect, the invention provides a method for administering at least one phospholipid composition to a subject, comprising: i) coating a medical device with at least one layer of the phospholipid composition, wherein the coating is achieved by electrospraying the device with the composition, and wherein the composition can carry at least one therapeutic agent; and ii) administering the coated device to the subject. In one embodiment of a method according to the invention, the composition is carrying at least one therapeutic agent.

In another embodiment of a method according to the invention, the medical device is selected from the group consisting of a cardiovascular device, an orthopedic device, an orthopedic fixture, an endoprosthetic, a stent, a graft, and an implant. In still another embodiment, the phospholipid is selected from phosphatidylcholine and phosphatidylserine.

In a further embodiment of a method according to the invention, the therapeutic agent is selected from a group consisting of an anti-bacterial agent, an angiogenic factor, an anti-cancer agent, and an anti-thrombogenic agent. In still another embodiment, the therapeutic agent is an antibiotic. The antibiotic may, for example, without limitation, be gentamicin or vancomycin.

In one embodiment of a method according to the invention, the phospholipid composition carries the therapeutic agent prior to coating the device. In another embodiment, the therapeutic agent is administered to the device after coating.

In one embodiment of a method according to the invention, the medical device is coated with more than one layer of a phospholipid composition. Each layer can, in a further embodiment, be a different phospholipid composition, i.e, comprising different phospholipid(s) and, optionally, different additive(s). Furthermore, each layer may, in another embodiment, carry a different therapeutic agent.

In one embodiment of a method according to the invention, the phospholipid composition is a solution of a suitable solvent carrying at least one phospholipid. For example, in one specific embodiment, the suitable solvent is chloroform.

In another embodiment of a method according to the invention, the medical device is treated prior to coating with an agent that enhances the adherence of the coating to the device. Such an agent may, for example, be calcium chloride or calcium phosphate, but is not limited to the same. In still another embodiment, the phospholipid composition further comprises an agent that enhances the elution profile and/or durability of the composition. Such an agent may, for example, be cholesterol, but is not limited to the same.

In one embodiment of a method according to the invention, at least one of the phospholipid, phospholipid concentration in the composition, amount of therapeutic agent, inclusion of additional additives in the composition, density of the layer, spray distance, voltage, and syringe pump rate are selected in order to adjust the elution profile for the therapeutic agent upon insertion/implantation in the subject.

In another aspect, the invention provides a kit for administering at least one phospholipid composition to a subject, comprising a medical device, at least one phospholipid composition for coating the device via electrospraying, wherein the composition is carrying or can carry at least one therapeutic agent, and instructions for use in conjunction with an electrospraying unit.

In still another aspect, the invention provides a medical device, wherein the medical device is coated via electrospraying with at least one layer of a phospholipid composition, wherein the composition can carry or is carrying at least one therapeutic agent, wherein the coating is substantially uniform and is less than or equal to about 20 microns thick. In one embodiment, the coating comprises multiple layers of phospholipid composition(s). Electrospraying one layer of phospholipid composition may result in a substantially uniform layer less than or equal to about 6 microns thick.

Other aspects of the invention are described in or are obvious from the following disclosure and are within the ambit of the invention.

### BRIEF DESCRIPTION OF THE DRAWINGS

The following Detailed Description of the Invention, given by way of Examples, but not intended to limit the invention to specific embodiments described, may be understood in conjunction with the accompanying figures, in which:

FIG. 1 graphically depicts percentage gentamicin sulfate released from 10 kV E-sprayed DOPS coating.

FIG. 2 graphically depicts percentage gentamicin sulfate released from 12 kV E-sprayed DOPS coating.

FIG. 3 graphically depicts percentage gentamicin sulfate released from 14 kV E-sprayed DOPS coating.

FIG. 4 shows, in bar graph form, gentamicin sulfate loading efficiencies, comparing three E-spray voltages, with 14 kV being significantly greater than for other voltages.

FIG. 5 graphically depicts total femoral infection, as measured by total luminescent flux. Lower flux indicates lower infection. Gentamicin sulfate-loaded DOPS coatings show significantly lower infection, which cleared in 15 days.

FIG. 6 graphically depicts percentage gentamicin sulfate released from enhanced coatings (test samples were pre-treated with calcium and E-sprayed with DOPS and cholesterol).

FIG. 7 shows, in bar graph form, a comparison of mean loading efficiencies, with DOPS/gentamicin sulfate on Ca-pre-treated with cholesterol significantly higher (p<0.05) than only DOPS/gentamicin sulfate.

FIGS. 8A and 8B show one graph each depicting the total flux over time of osteomyelitis infection in mice undergoing gentamicin treatment. Values of total flux below 3E+04 indicate no presence of infection. FIG. 8A tracks all ten mice for all treatments. For FIG. 8B, three mice were removed from the DOPS+gentamicin sulfate (GS) group as potential outliers based on potential technical difficulties.

### DETAILED DESCRIPTION

### Definitions

The term “medical device” as used herein includes devices, implants, and grafts and refers to man-made, synthetic implantable materials (e.g., metals, plastics, polymers, ceramics, composites, semiconductors) or biological implantable materials (e.g., autografts, allografts, xenografts) that can be administered to or placed in or inserted into or grafted into the body of a subject to correct a clinical condition or for prosthetic, therapeutic, diagnostic, or experimental purposes. The medical device is either electrically conductive or can be rendered conductive prior to electro-spraying (for example, by coating the device with a conductive fluid that does not, however, interfere with the bonding between the coating composition and the device).

The term “therapeutic agent” as used herein refers to an agent capable of producing a therapeutic effect or an inductive effect or assisting in diagnosis.

The term “substantially uniform” as used herein refers to the E-sprayed phospholipid composition coating on the medical device being of about the same thickness on about every surface of the device, wherein the surface includes the holes, pockets, and other features on the device's inner and outer surfaces. In one embodiment of the invention, the term “substantially uniform” indicates that at least about 70% of the surface of the medical device has been coated. In further embodiments of the invention, the term “substantially uniform” indicates that at least about 80% of the surface of the medical device has been coated, that at least about 90% of the surface of the medical device has been coated, that at least about 95% of the surface of the medical device has been coated, that at least about 96% of the surface of the medical device has been coated, that at least about 97% of the surface of the medical device has been coated, that at least about 98% of the surface of the medical device has been coated, that at least about 99% of the surface of the medical device has been coated, or that at least about 100% of the surface of the medical device has been coated.

The term “carry” as used herein refers to incorporate or comprise.

The term “subject” as used herein refers to a vertebrate, preferably a mammal. Mammals include, without limitation, primates (humans, gorillas, monkeys, apes), wild animals, feral animals, farm animals (porcine, bovine, ovine sheep, equine), sports animals, and pets (for example, horse, dog, cats).

The terms “comprises”, “comprising”, are intended to have the broad meaning ascribed to them in U.S. Patent Law and can mean “includes”, “including” and the like. Moreover, the use of the term “including”, as well as other forms, such as “includes” and “included”, is not limiting.

The invention can be understood more fully by reference to the following detailed description and illustrative examples, which are intended to exemplify non-limiting embodiments of the invention.

### Embodiments of the Invention

Medical Device

A medical device employed in a method according to the invention may be selected, for example, from the group consisting of an orthopedic device, an orthopedic fixture, and an endoprosthetic. Pacemakers and defibrillators are likewise contemplated as medical devices employed in a method of the invention. In additional embodiments, the medical device may be a stent, such as a cardiovascular stent, or a graft (such as a bone graft, including an autograft or an allograft), such as a cardiovascular graft (e.g., a blood vessel graft). Common synthetic grafts are ceramic-based (calcium phosphate or calcium sulfate), Bioglass, or polymer-based materials or combinations of the two. A medical device for coating according to a method of the invention may be any conductive or rendered conductive device.

Synthetic implants such as breast, penile, dental, and cochlear implants and synthetic devices such as knee, hip, and shoulder prostheses, all contemplated as medical devices employable in a method according to the invention, are commonly made of metal, ceramic, and polymer plastics. The exact components depend on the type of implant or device, its intended location of the body, and even the health and immunological response of the individual.

The medical device may, in one embodiment, be pre-treated with an agent to customize particular coating adherence characteristics to the device in order to engineer specific applications of the coating. In a specific embodiment, the medical device is treated with calcium chloride or calcium phosphate before coating with the phospholipid composition.

The pre-treatment of the medical device can, for example, be achieved via coating, passivation, or other known surface modification methods, i.e., methods that modify the surface of the device to alter its roughness, hydrophilicity, surface charge, surface energy, biocompatibility, and/or reactivity, or surface functionalization methods, i.e., methods that introduce chemical functional groups to the surface of the medical device.

Titanium sees broad clinical use in orthopedic, dental and craniofacial applications due to its biocompatibility, mechanical properties and corrosion resistance. Bone healing around titanium implants is characterized by a gradual mineralization process, directed from surrounding tissue toward the implant.

Stainless steel, cobalt chrome, and titanium alloys (for example, titanium medical alloy Ti-6Al-4V) are likewise commonly used metals in implants.

Therapeutic Agent

It is contemplated that many different therapeutic agents can be administered using a method according to the invention. Therapeutic agents can be selected from the group consisting of, but not limited to, anesthetic agents, antiarrhythmic agents, antihypertensive agents, anti-inflammatory agents, antibacterial agents, antiparasitic agents, chemotherapeutic agents (for example, carboplatin or another alkylating agent), coupling agents, antiadrenergic agents, antianginal agents, sympathetic agents, and therapeutic proteins.

Growth factors may, in turn, be selected as the therapeutic agent to promote recellularization, vascularization, or epithelialization. Other so-called recellularization agents include, without limitation, chemoattractants, cytokines, chemokines, and derivatives thereof.

In a specific embodiment of a method according to the invention, the therapeutic agent is an antibacterial agent. The antibacterial agent may, for example, be an antibiotic. A wide range of antibiotics are used to treat osteomyelitis and other infections common in orthopedic surgery. Of these, the aminoglycoside antibiotics, such as streptomycin, tobramycin and gentamicin, exhibit bactericidal activity against a broad range of microorganisms.

In another specific embodiment, a phospholipid composition coating layer may carry more than one therapeutic agent.

It is additionally contemplated that the medical device can be coated with multiple layers of distinct coating (phospholipid) compositions. Each respective coating composition (i.e., each respective layer) could have either the same therapeutic agent or a different therapeutic agent or even a combination of therapeutic agents, as is described in greater detail below.

Coating Composition

The composition for coating the medical device comprises a solution of a suitable solvent carrying at least one phospholipid. Many solvents can be phospholipid carriers, depending on the intended application of the composition and the phospholipids(s) to be included in the solvent. In one embodiment, the solvent is chloroform. Chloroform is a non-polar solvent. Other non-polar solvents, polar aprotic solvents, polar protic solvents, and water may also be used as phospholipid carriers.

In one embodiment of a method of the invention, the phospholipid composition E-sprayed onto a medical device does not carry any therapeutic agent. The phospholipid(s) of the composition may or may not exert its own therapeutic effect upon administration to insertion or implantation of the coated medical device into a subject.

Phospholipids are widely used as drug delivery vehicles. Their amphiphilic composition and molecular complexity provides them with a wide range of chemical bonding options, thus enabling them to carry a wide range of therapeutics. The majority of phospholipid-derived drug delivery configurations found in the literature make use of lipid meta-structures such as liposomes, microspheres, micelles, and reverse micelles.

The phospholipid may be natural or synthetic. In one embodiment, the phospholipid is selected from the group consisting of phosphatidic acid, phosphatidylethanolamine, phosphatidylcholine, phosphatidylserine, phosphatidyl glycerol, phosphotidylinositol, phosphotidylinositol phosphate, phosphotidylinositol biphosphate, phosphotidylinositol triphosphate, ceramide phosphorylcholine, ceramide phosphorylethanolamine, and ceramide phosphorylglycerol. In a specific embodiment, the phospholipid is selected from phosphatidylcholine and phosphatidylserine.

The phospholipid composition for coating the medical device may, in one embodiment, comprise more than one phospholipid, for example, a mixture of phosphatidylcholine and phosphatidylserine.

Phospholipid concentration in the phospholipid composition for coating a medical device via E-spraying according to a method of the invention may be between about 0.1% and about 10% w/v, preferably between about 0.5% and about 7% w/v. The exact preferred range of phospholipid concentration will vary with the phospholipid (or mixture of phospholipids) employed.

In one embodiment, the phospholipid is phosphatidylcholine. In another embodiment, the concentration of phosphatidylcholine is between about 1% and about 10% w/v, preferably between about 3% and about 6% w/v, more preferably between about 4% and about 5% w/v.

In a further embodiment, the phospholipid is phosphatidylserine. In still another embodiment, the concentration of phosphatidylserine is between about 0.1% and about 10% w/v, preferably between about 1% and about 5% w/v, more preferably between about 1% and about 2% w/v.

It is additionally contemplated that the phospholipid composition may comprise at least one additive. Such an additive would customize particular coating characteristics in order to allow the ordinarily skilled artisan to engineer specific applications of the coating. In one embodiment, the coating composition may additionally comprise cholesterol. Cholesterol increases the stability of phospholipids (Grit, M., et al. 1993 Chem Phys Lipids 64(1-3):3-18) and regulates membrane fluidity over the range of physiological temperatures (Yu, G., et al. 2006 J Mater Sci: Mater Med 17:899-909). Thus, the inclusion of cholesterol in the coating composition may augment its stability through the latter's interaction with phospholipids.

In another embodiment, the stability, durability, stiffness, and/or toughness of the coating composition may be increased by using synthetic phospholipids.

Administration: Electrospraying

The coating of the medical device with the phospholipid composition in a method according to the invention is achieved by electrospraying (E-spraying).

E-spraying can create thin coatings with high efficiencies (Siefert, W. 1984 Thin Solid Films 120(4):267-274), because the charged liquid source material is carried by the electrical field rather than being pressurized or carried on another liquid, as in typical pressure-based spraying techniques based on atomization of a liquid (Kumbar, S. G., et al. 2008 Biomed Mater 3(3)).

This is not only advantageous for more costly coating materials, but, more importantly, it enables good control of coating uniformity and morphology, especially on rough and intricately shaped surfaces (Chen, C. H., et al. 1999 Thin Solid Films 342(1-2):35-41). Finally, E-spraying provides relatively easy control of product stoichiometry and morphology.

E-spraying is also advantageous, because it allows the coating of 3-dimensional surfaces. Complex shapes, including shapes with pores, can be coated uniformly, even conformally, via E-spraying. This is a surprising advantage over conventional dip and drip techniques.

As such, E-spraying phospholipid compositions, optionally impregnated with one or more therapeutic agents, on an implant provides unexpected advantages over conventional coating techniques. Numerous E-spraying process parameters affect the creation of the surface on the target material (Hartman, R. P. A., et al. 1999 J Aerosol Sci 30(7):823-849; Uematsu, I., et al. 2004 J Colloid Interface Sci 269(2):336-340), including: concentration of the source liquid, syringe pump rate, size (surface area) of target material, distance from source to target, voltage potential, E-spray time (delivering a known amount of source liquid into the electric field at the pump rate), physical properties of the source liquid (surface tension, viscosity, density and electrical conductivity), and capillary (needle) diameter.

In addition, for the application of E-spraying targeted at creating coatings that would be used in drug delivery and cell growth studies, additional characteristics such as chemical composition of source material are also important considerations.

The syringe pump rate may, in one embodiment, be between about 5 mL/hr and about 20 mL/hr. In one embodiment, the phospholipid is phosphatidylcholine, and the syringe pump rate is between about 5 mL/hr and about 15 mL/hr, preferably between about 7 mL/hr and about 11 mL/hr, more preferably about 10 mL/hr.

In another embodiment, the phospholipid is phosphatidylserine, and the syringe pump rate is between about 10 mL/hr and about 20 mL/hr, preferably about 14 mL/hr.

The total target sample area may, in one embodiment, be between about 0.5 cm² to about 11 cm². In one embodiment, the phospholipid is phosphatidylcholine, and the total target sample area is between about 1 cm² and about 5 cm². The target refers to the medical device in the instant context.

In another embodiment, the phospholipid is phosphatidylserine, and the total target sample area is between about 3 cm² to about 10 cm².

The spray distance can, in one embodiment, be between about 5 cm and about 15 cm. In one embodiment, the phospholipid is phosphatidylcholine, and the spray distance is between about 10 cm and about 14 cm. In another embodiment, the spray distance is about 12 cm.

In another embodiment, the phospholipid is phosphatidylserine, and the spray distance is between about 6 cm and about 10 cm. In another embodiment, the spray distance is about 8 cm.

The voltage can, in one embodiment, be between about 5 kV and about 20 kV. In one embodiment, the phospholipid is phosphatidylcholine, and the voltage is between about 8 kV and about 18 kV. In another embodiment, the voltage is about 10 kV.

In another embodiment, the phospholipid is phosphatidylserine, and the voltage is between about 8 kV and about 12 kV. In another embodiment, the voltage is about 12 kV.

The spray time can, in one embodiment, be between about 30 seconds and about 10 minutes. In one embodiment, the phospholipid is phosphatidylcholine, and the spray time is between about 3 minutes and about 10 minutes.

In another embodiment, the phospholipid is phosphatidylserine, and the spray time is between about 30 seconds and about 5 minutes.

The ranges provided above are not meant to be limiting.

Elution Profile

The phospholipid(s) selected, the phospholipid concentration in the coating composition, the amount of therapeutic agent, the inclusion of additional additives in the phospholipid coating composition, and the E-spraying parameters discussed herein may be set or changed in order to adjust the elution profile for the therapeutic agent upon insertion/implantation of the coated medical device in the subject. The elution profile refers to the rate of elution of the therapeutic agent, as well as the shape of the elution curve (for example, a faster initial rate, followed by a slower rate and, finally, a faster rate; or a bolus some number of days or weeks out).

For example, coating a medical device (via E-spraying) with a phospholipid composition, wherein the phospholipids carry an antibiotic, may show that the antibiotic has eluted off within a few hours. In comparison, coating the device with the phospholipid composition additionally comprising cholesterol may show more than 50% of the antibiotic left after eight hours. In another example, a medical device may be coated (via E-spraying) with multiple phospholipid composition layers, wherein the first layer (closest to the device) may be quite dense and hold onto its therapeutic agent longer, the second layer may be less dense (carrying, for example, growth factor), and its third layer thin (carrying, for example, a high concentration of antibiotic).

For example, for an antibiotic such as gentamicin, typical elution profiles are characterized by an initial release “burst”, the size of which is proportional to the concentration of the antibiotic, followed by a gradual release over 3 to 50 days. The particular material carrying the gentamicin has a direct affect on the elution profile (initial burst, rate of release, and decay).

Administration: Other

In one embodiment of a method according to the invention, the medical device is coated via E-spraying with a phospholipid composition that can, but does not, carry at least one therapeutic agent. In this embodiment, the therapeutic agent, if one is to be administered at all, can be administered to the coated device at a later time. The agent may, for example, be administered to the coated device via dip-coating or drip-coating.

In another embodiment of a method according to the invention, the medical device is coated via E-spraying with a phospholipid composition that carries at least one therapeutic agent. It is, however, also contemplated that at least one additional therapeutic agent can be administered to the coated device at a later time. The additional agent may, for example, be administered to the coated device via dip-coating or drip-coating.

In still another embodiment of a method according to the invention, the medical device is coated via E-spraying with a phospholipid composition carrying at least one therapeutic agent. The coated device can subsequently be coated via E-spraying with at least one additional phospholipid composition carrying or that can carry at least one additional therapeutic agent. If the additional composition is not carrying an additional therapeutic agent upon E-spraying, an additional therapeutic agent can be administered to the multiply coated device at a later time. The additional agent may, for example, be administered to the multiply coated device via dip-coating or drip-coating. Consequently, a singly or multiply coated device resulting from a method according to the invention will have some defined shelf-life and certain arbitrary storage conditions determined by the makeup of the coating composition(s).

These embodiments allow for so-called custom coating. The device could be coated with a phospholipid composition carrying a therapeutic agent with a specific patient or patient population in mind Likewise, the device could be coated with a phospholipid composition that can carry, but does not yet, a therapeutic agent. The device could then have a desired therapeutic agent administered to it upon determination of the desired treatment course for a patient. The administration of the therapeutic agent or additional coating with additional phospholipid composition(s) carrying or that can carry another therapeutic agent could be carried out in or near the treatment facility, even in an operating room.

Kits

In one aspect, the invention provides kits for administering a therapeutic agent to a subject, comprising a medical device for coating via E-spraying, at least one phospholipid composition, wherein the composition is carrying or can carry at least one therapeutic agent, and instructions for use in conjunction with an E-spraying unit.

A kit according to the invention can further comprise an E-spraying unit.

Alternatively, the invention provides kits for administering a phospholipid composition to a subject, comprising a medical device for coating via E-spraying, at least one phospholipid composition, wherein the composition can carry at least one therapeutic agent, and instructions for use in conjunction with a medical device and an E-spraying unit. For example, the kit could be used in conjunction with the treatment regimen of a subject. A medical device could be electrosprayed with a phospholipid composition in the kit and then, upon drying, inserted into the patient. Alternatively, a medical device could be electrosprayed with a phospholipid composition in the kit and then, upon drying, be subjected to drip-coating with at least one desired therapeutic agent, followed by administration to or insertion into the patient when dry.

A kit according to the invention can also comprise materials for storage of the medical device after E-spray coating it with the phospholipid composition, so that the coated device would be ready for later administration of a therapeutic agent and administration to or insertion into a subject.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

### EXAMPLES

The following examples are provided for illustrative purposes only and are not intended to limit the scope of the invention.

### Example 1

The following combinations of E-spray process parameters were tested with phosphatidylcholine:
1 cm×1 cm square, flat, commercially pure titanium samples
1,2-Distearoyl-sn-glycero-3-phosphocholine (DSPC) concentrations of 4% and 5% (mass %)
Pump rates ranging from 7 ml/hr to 11 ml/hr
Total target sample areas ranging from 1 cm² to 5 cm²
Spray distances ranging from 10 cm to 14 cm
Voltages ranging from 8 kV to 18 kV
Spray times ranging from 3:30 minutes to 25 minutes
22 gauge needle diameter.

E-spray parameters for DSPC E-spray coating were found to be especially effective, for example, at: DSPC concentration: 5% (w/v)
Pump rate: 10 ml/hr
Spray time: 4 minutes per set of samples
Spray distance: 12 cm
Voltage: 10 kV

### Example 2

The following combinations of parameters were tested with phosphatidylserine (DOPS):
0.5 cm×0.5 cm square, flat, commercially pure titanium samples
1,2-di-(9Z-octadecenoyl)-sn-glycero-3-phospho-L-serine (DOPS) concentrations ranging from 0.6% to 6% (w/v))
Pump rates ranging from 10 ml/hr to 20 ml/hr
Sample square areas ranging from 3 cm² to 10 cm²
Spray distances ranging from 6 cm to 10 cm
Voltages ranging from 8 kV to 12 kV
Spray times from 36 seconds to 9:15 minutes

E-spray parameters for DOPS E-spray coating were found to be especially effective, for example, at: DOPS concentration: 1.3% (w/v)
Pump rate: 14 ml/hour
Spray distance: 8 cm
Voltage: 12 kV
Spray time: 3 minutes per set of samples.

### Example 3A

A solid titanium medical device (e.g. a hip stem or endoprosthesis) is coated (via E-spraying) with multiple phospholipid composition layers, wherein the first layer (closest to the device) carries an antibiotic and is quite dense and sticky, e.g., is combined with relatively large amounts of cholesterol to hold the antibiotic relatively longer, e.g., for release in 30 days to eradicate any lingering microbes. The second layer is less dense, and carries a growth factor substance to motivate bone growth. The third, outermost layer is relatively thin and carries a relatively high dosage of an antibiotic, in quick-releasing and quickly dispersing form, for eradication of initial microbes at time of surgery. Table 1, below, provides exemplary E-spraying parameters.

### Example 3B

A titanium alloy foam or lattice medical device is coated (via E-spraying) with multiple phospholipid composition layers, wherein the first layer (deepest penetrating into the device, i.e., E-sprayed at the highest voltage) carries an angiogenic factor (i.e., to motivate blood vessel growth), and the second layer, penetrating less deeply into the device, carries a growth factor substance for motivating bone growth. The third, outermost layer carries a relatively high dosage of an antibiotic, in quick-releasing and quickly dispersing form, for eradication of initial microbes at time of surgery. These coatings are applied in the operating room during surgery by the physician, who estimates dosages and implant shape “in theatre”. Table 1, below, provides exemplary E-spraying parameters.

### Example 3C

A medical device (e.g., a catheter) is coated (via E-spraying) with multiple phospholipid composition layers, wherein the first layer (closest to the device) carries an anti-coagulant to aid in resisting coagulation of blood in the catheter, while the second layer carries an anti-inflammatory therapeutic agent to counter natural inflammation of the wound area, and the third layer carries a relatively low dosage of antibiotic, in quick-releasing and quickly dispersing form, for eradication of initial microbes at time of insertion through the patient's skin. Table 1, below, provides exemplary E-spraying parameters.

### Example 3D

A medical device consisting of the patient's own bone (i.e., autograft) is coated (via E-spraying) with multiple phospholipid composition layers, wherein the face of the device that is to be inserted facing into the patient is coated with an angiogenic factor (i.e., to motivate blood vessel growth). The second layer is less dense and carries a growth factor substance to motivate bone growth. The third layer carries a slow releasing antibiotic for longer-term defense against microbes, and a fourth, outermost layer that is relatively thin carries a relatively high dosage of an antibiotic, in quick-releasing and quickly dispersing form, for eradication of initial microbes at time of surgery. The face of the device that is to be inserted facing outward from the patient is coated (via E-spraying) with multiple phospholipid composition layers, wherein the first layer (closest to the device) carries a growth factor substance for motivating bone growth. And a second, outermost layer carries a relatively high dosage of an antibiotic, in quick-releasing and quickly dispersing form, for eradication of initial microbes at time of surgery. These coatings are applied in the operating room during surgery by the physician, who estimates dosages and implant shape “in theatre”. Table 1, below, provides exemplary E-spraying parameters.

### Example 3E

A medical device referred to as a cardiovascular stent is coated (via E-spraying) with multiple phospholipid composition layers, wherein the first layer (closest to the device) carries an anti-thrombogenic polymer to counter potential thrombus (i.e., blood clot) formation due to damage to passing blood cells, while the second layer carries two distinct therapeutic agents, an anti-inflammatory agent to counter natural inflammation at the insertion site, along with a relatively high dosage of an antibiotic, in quick-releasing and quickly dispersing form, for eradication of initial microbes at time of surgery. Table 1, below, provides exemplary E-spraying parameters.

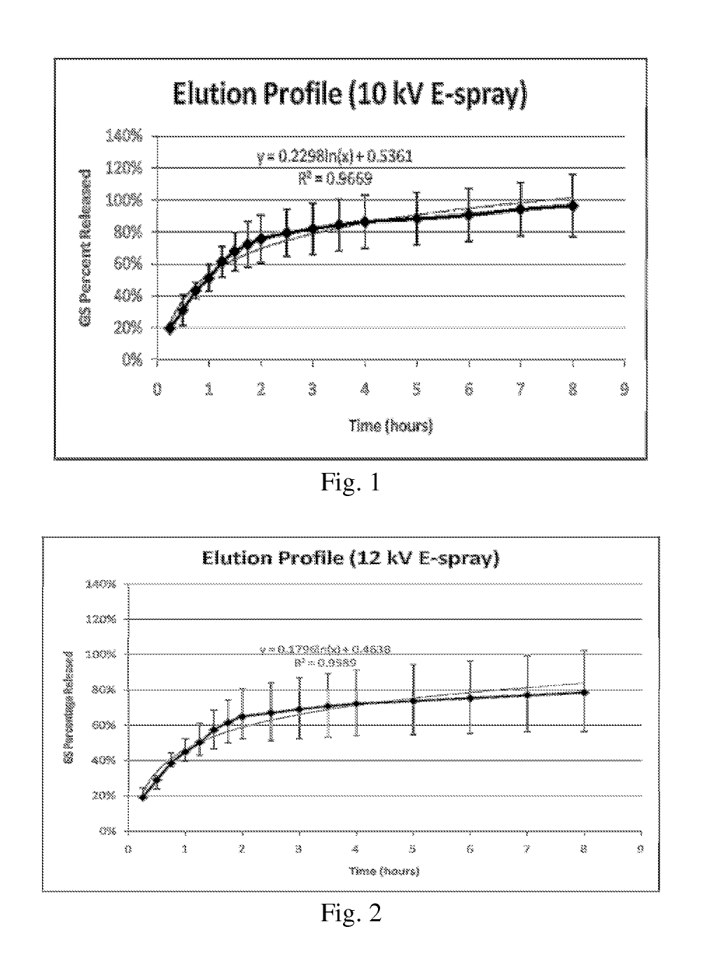

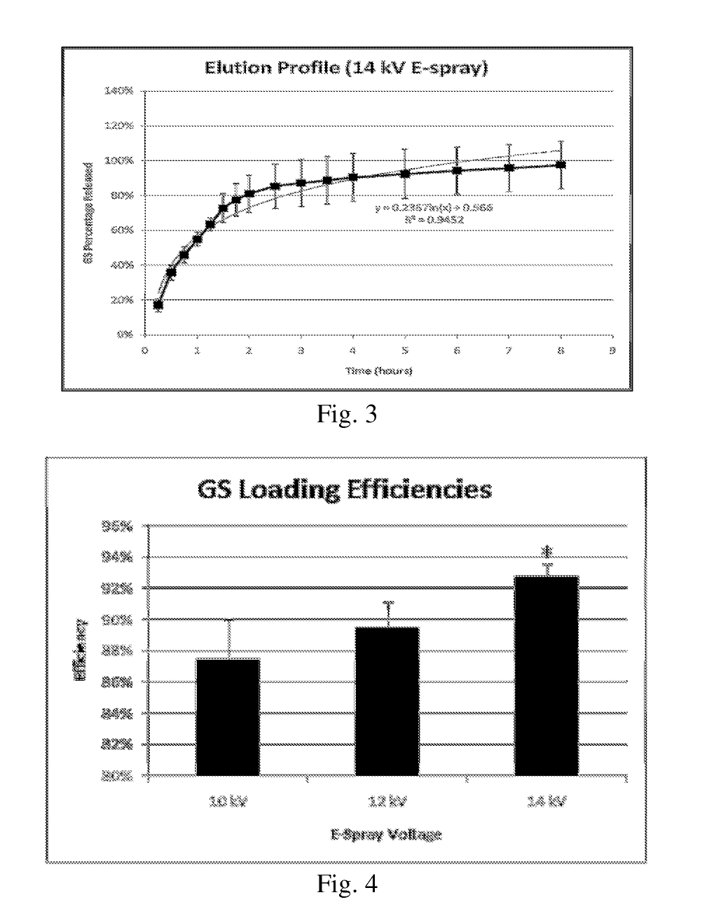

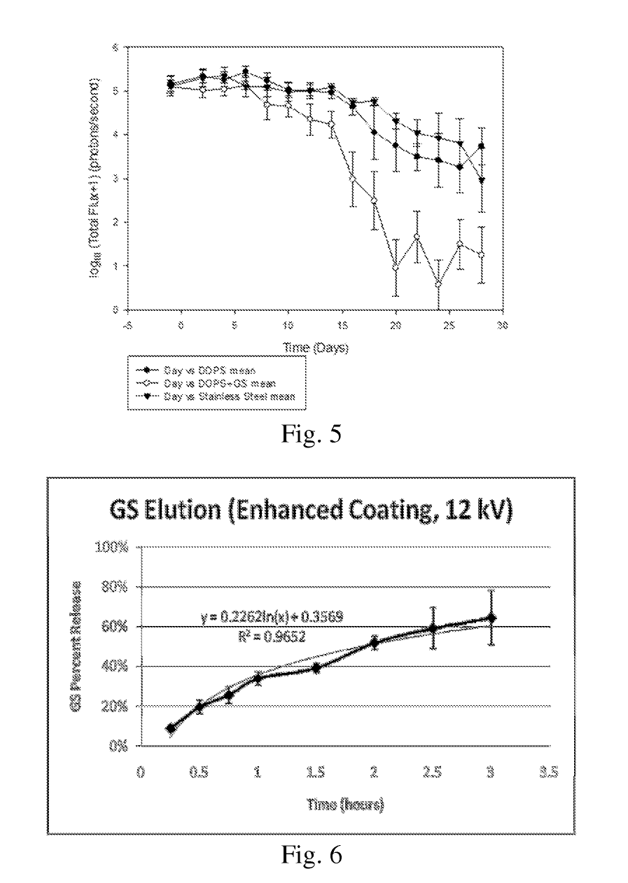

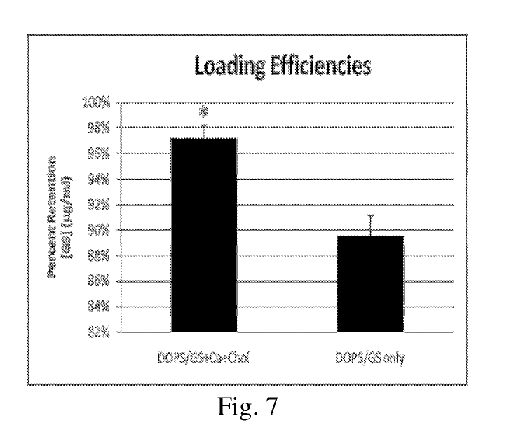

TABLE 1
exemplary, non-limiting E-spray parameters:

| | | Electrospraying Parameters for a | |
|---|---|---|---|
| Electrospraying | Ingredient | Ti Implant and Potential Elution | |
| Composition | Parameters | Profile where Appropriate | |
| Phospholipid | Phosphaditylcholine | Pump rate: 10 ml/hr | |
| composition not | (DSPC): 5% (w/v) | Spray time: 4 minutes delivering | |
| carrying | (single layer) | 19.4 mg of DSPC per cm² sample | |
| therapeutic | | area | |
| agent E-sprayed | | Spray distance: 12 cm | |
| on titanium | | Voltage: 10 kV | |
| | Phosphatidylserine | Pump rate: 14 ml/hour | |
| | (DOPS): 1.3% (w/v) | Spray time: 3 minutes per set of | |
| | (other potential | samples delivering 1.2 mg of DOPS | |
| | phospholipids) | per sample, in this case 3 samples | |
| | (2 layers of DOPS) | producing a coverage of 3.2 | |
| | | mg/cm². | |
| | | Spray distance: 8 cm | |
| | | Voltage: 12 kV | |
| Phospholipid | Phosphatidylserine | Pump rate: 14 ml/hour | |
| composition | (DOPS): 1.3% (w/v) | Spray time: 3 minutes per set of | |
| carrying | Gentamicin (GS) | samples delivering 1.2 mg of DOPS | |
| therapeutic | Load: 400 μg | per sample, in this case 3 samples | |
| agent on | (other potential | producing a coverage of 3.2 | |
| titanium | agents, e.g. | mg/cm². | |
| | antibacterial, anti- | Spray distance: 8 cm | |
| | cancer, etc.) | Voltage: 10 kV | |
| | (2 layers | 8 hour elution | |
| | sandwiching GS | (FIGS. 1, 4) | |
| | layer: DOPS, then | Voltage: 12 kV | |
| | GS, then DOPS) | 8 hour elution | |
| | | (FIGS. 2, 4) | |
| | | Voltage: 14 kV | |
| | | 8 hour elution | |
| | | (FIGS. 3, 4) | |
| Phospholipid | Phosphatidylserine | Pump rate: 14 ml/hour | Infection |
| composition | (DOPS): 1.3% (w/v) | Spray time: 3 minutes per set of | Eradication |
| carrying | Gentamicin (GS) | needles | (flux~infection) |
| therapeutic | Load: 270 μg | Spray distance: 8 cm | (FIG. 5) |
| agent on | (other potential | Voltage: 14 kV | |
| stainless steel | agents, e.g. | | |
| | antibacterial, anti- | | |
| | cancer, etc.) | | |
| | (2 layers | | |
| | sandwiching GS | | |
| | layer: DOPS, then | | |
| | GS, then DOPS) | | |
| Phospholipid | Phosphatidylserine | Pump rate: 14 ml/hour | |
| composition | (DOPS): 1.3% (w/v) | Spray time: 3 minutes per set of | |
| with cholesterol | Gentamicin (GS) | samples delivering 1.2 mg of DOPS | |
| as additive | Load: 200 μg | per sample, in this case 3 samples | |
| | (other potential | producing a coverage of 3.2 | |
| | antibacterial, anti- | mg/cm². | |
| | cancer, etc. agents) | Spray distance: 8 cm | |
| | (2 layers | Voltage: 12 kV | |
| | sandwiching GS | 3 hour elution | |
| | layer: DOPS, then | (FIGS. 6, 7) | |
| | GS, then DOPS) | | |

### Example 4

Osteomyelitis was induced in Black-6 mice with bacteria transduced to express luciferase. This allowed for tracking of the infection without sacrificing the mice. 22 gauge stainless steel needles pre-treated with calcium chloride were coated (via E-spraying) with a 6:1 DOPS:cholesterol phospholipid composition (with or without gentamicin sulfate) were inserted as intramedullary implant, and the response to the implants was observed over time. E-spray parameters were DOPS concentration 1.3% (w/v), 14 kV, pump rate 14 mL/hr, spray distance 8 cm, 45 seconds spray time per sample set, loaded with 270 μg gentamicin.

Serum and urine were taken from the mice at the end of the study. The serum was analyzed for blood urea nitrogen (BUN) and creatinine levels. The urine was analyzed for protein content.

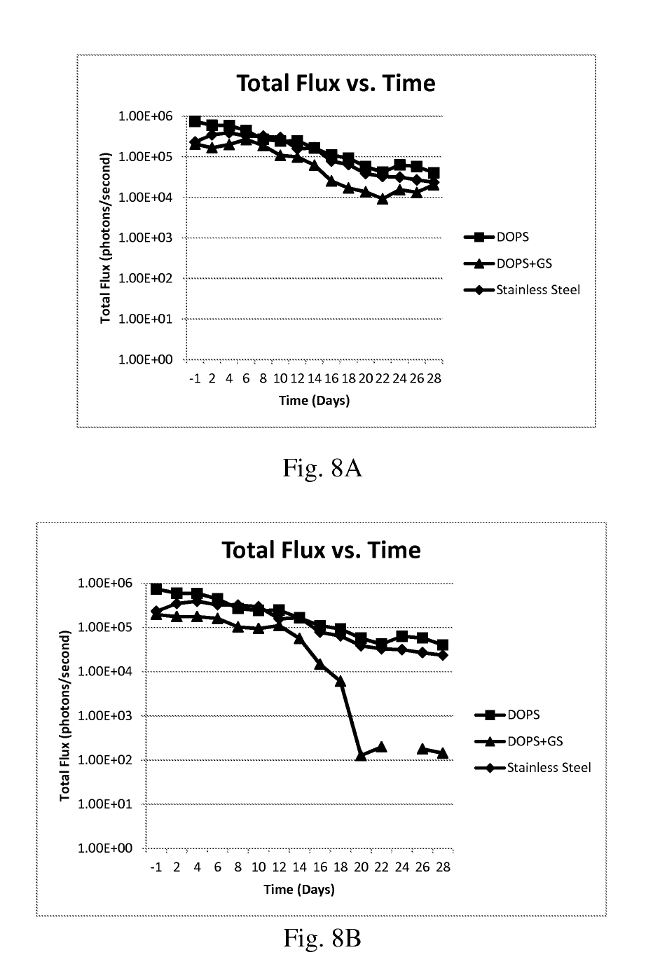

The implants loaded with antibiotic were able to clear the infection faster than the other treatment groups (FIG. 8). Creatinine and urine protein values showed no significant differences between the treatment groups. BUN levels were higher for implants with antibiotic than the other groups, but still appeared to be within a normal range, indicating that the implants did not result in kidney damage.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

What is claimed is:

1. A method for administering at least one phospholipid composition to a subject, comprising: i) coating a medical device with at least one layer of the phospholipid composition, wherein the coating is achieved by electrospraying the device with the composition, and wherein the composition can carry at least one therapeutic agent; and ii) administering the coated device to the subject.

2. The method of claim 1, wherein the composition is carrying at least one therapeutic agent.

3. The method of claim 1, wherein the medical device is selected from the group consisting of a cardiovascular device, an orthopedic device, an orthopedic fixture, an endoprosthetic, a stent, a graft, and an implant.

4. The method of claim 1, wherein the phospholipid is selected from phosphatidylcholine and phosphatidylserine.

5. The method of claim 2, wherein the therapeutic agent is selected from a group consisting of an anti-bacterial agent, an angiogenic factor, an anti-cancer agent, and an anti-thrombogenic agent.

6. The method of claim 5, wherein the therapeutic agent is an antibiotic.

7. The method of claim 6, wherein the antibiotic is gentamicin or vancomycin.

8. The method of claim 2, wherein the phospholipid composition carries the therapeutic agent prior to coating the device.

9. The method of claim 2, wherein the therapeutic agent is administered to the device after coating.

10. The method of claim 1, wherein the medical device is coated with more than one layer of a phospholipid composition.

11. The method of claim 2, wherein the medical device is coated with more than one layer of a phospholipid composition.

12. The method of claim 11, wherein each layer is carrying or can carry a different therapeutic agent.

13. The method of claim 1, wherein the phospholipid composition is a solution of a suitable solvent carrying at least one phospholipid.

14. The method of claim 1, wherein the medical device is treated prior to coating with an agent that enhances the adherence of the coating to the device.

15. The method of claim 14, wherein the agent is calcium chloride or calcium phosphate.

16. The method of claim 1, wherein the phospholipid composition further comprises an agent that enhances the elution profile and/or durability of the composition.

17. The method of claim 16, wherein the agent is cholesterol.

18. The method of claim 2, wherein at least one of the phospholipid, phospholipid concentration in the composition, amount of therapeutic agent, inclusion of additional additives in the composition, density of the layer, spray distance, voltage, and syringe pump rate are selected in order to adjust the elution profile for the therapeutic agent upon insertion/implantation in the subject.

19. A kit for administering at least one phospholipid composition to a subject, comprising a medical device, at least one phospholipid composition for coating the device via electrospraying, wherein the composition is carrying or can carry at least one therapeutic agent, and instructions for use in conjunction with an electrospraying unit.

20. A medical device, wherein the medical device is coated via electrospraying with at least one layer of a phospholipid composition, wherein the composition can carry or is carrying at least one therapeutic agent, wherein the coating is substantially uniform and is less than or equal to about 20 microns thick.
